(19) United States
(12) Patent Application Publication
(10) Pub. No.: US 2018/0161592 A1
(43) Pub. Date: Jun. 14, 2018

(54) CONTACT-TYPE LASER OPERATING HEAD AND DEVICE FOR USE IN MEDICAL TREATMENT AND COSMETOLOGY WITH LASER

(71) Applicant: Focuslight Technologies Inc., XI'AN (CN)

(72) Inventor: Ben Meng, XI'AN (CN)

(73) Assignee: Focuslight Technologies Inc., XI'AN (CN)

(21) Appl. No.: 15/571,032

(22) PCT Filed: Sep. 28, 2016
(86) PCT No.: PCT/CN2016/100532

§ 371 Date: Oct. 31, 2017

(30) Foreign Application Priority Data
Sep. 3, 2015 (CN) 201510641067.8

(51) Int. Cl. A61N 5/06; G02B 6/42

(52) U.S. Cl. CPC A61N 5/0616; A61N 2005/005; G02B 6/4268; G02B 6/4204

(57) ABSTRACT

Disclosed are a contact-type laser operating head and a device for use in medical treatment and cosmetology with laser. The contact-type laser operating head mainly comprises a light emitting port fixture (8) and several light emitting modules forming a light emitting port. The light emitting modules on the periphery and the light emitting port fixture (8) as well as the adjacent light emitting modules are connected by connectors (7). The connector (7) itself or an adaptive connecting structure thereof enables the light emitting modules to be driven to rotate independently so as to follow and attach to the surface to be treated. The light emitting module comprises a lens fixing frame (5) and an optical fibre fixture (6), wherein the lens fixing frame (5) is used to mount a functional lens (2), and the optical fibre fixture (6) is fixedly mounted behind the lens fixing frame (5) for packaging a coupled optical fibre (4) and keeping a light beam output from the optical fibre (4) perpendicular to a lens surface. The contact-type laser operating head can be effectively attached to an irregular surface to be treated, thereby improving the working efficiency, and reducing the danger of laser leakage.

### TECHNICAL FIELD

The disclosure herein relates to the field of laser medical cosmetology, particularly relates to a contact-type laser operating head and a device for use in medical treatment and cosmetology with laser, which are suitable for medical cosmetology, etc.

### BACKGROUND

An existing product uses one light emitting port to serve as a treatment light emitting port. Since the skin of the human body has angular corner locations, the light emitting port is relatively small, the treatment efficiency is low, there is the risk of light leakage when the angular corner locations cannot be tightly fitted with the skin.

### SUMMARY

Disclosed herein is a novel contact-type laser operating head, which can fit irregular surface to be processed, improve working efficiency, and reduce the risk of light leakage.

The technical scheme of this invention is as follows:

A contact-type laser operating head mainly comprises a light emitting port fixture and a plurality of light emitting modules forming a light emitting port, wherein a peripheral light emitting module on a periphery of the plurality of light emitting modules and the light emitting port fixture as well as the adjacent light emitting modules are configured to be connected by connectors; wherein the connectors or an adaptive connecting structure thereof enables each of the plurality of light emitting modules to be driven to rotate independently so as to follow and fit a to-be-processed surface; wherein the light emitting module comprises a lens fixing frame and an optical fiber fixture, wherein the lens fixing frame is configured to be used to mount a functional lens, and the optical fiber fixture is configured to be fixedly mounted behind the lens fixing frame, for packaging a coupling optical fiber and keeping a light beam output from the optical fiber to be perpendicular to the mirror surface.

According to different laser processing requirements, the “functional lens” herein can have different choices, for example, using a single-sided polished frosted glass to homogenize the light spots, using a concave mirror to achieve the function of expanding the light beam and enlarge the light spot, using a convex mirror to achieve the focusing effect and achieve the function of decreasing the light spot. The “peripheral light emitting module” herein is determined according to the specific light emitting module distribution mode, it can be all the light emitting modules (as shown in FIG. 5 and FIG. 6), and can also be partial light emitting modules (as shown in FIG. 7).

On the basis of the above scheme, the invention further provides the following optimization:

The optical fiber fixture is a conical frame structure, and an optical fiber coupling interface is arranged on the conical top.

The first type of structure for the connector: a corresponding connecting part between the connector and the light emitting port fixture, and a corresponding connecting part between the connector and the light emitting module, both form spherical auxiliary components.

The scheme for such a spherical auxiliary component, can be specifically divided as follows:

1. The main body of the connector is a rigid connecting rod, the two ends of the rigid connecting rod are spherical parts, a spherical concave hole is formed in the periphery of the lens fixing frame of the light emitting module, and is connected with the spherical part in an adaptive mode;

2. The main body of the connector is a rigid connecting block, the two ends of the rigid connecting block are spherical concave holes, the spherical connector which extends out from the outer periphery of the lens fixing frame of the light emitting, is connected with the spherical concave hole in an adaptive mode;

The second type structure of the connector: 3. The connector itself is a connecting rod made of high-strength elastic material (preferably polytetrafluoroethylene), the strength and the elasticity should meet the requirement of the independently forced rotation of every light emitting module. So it is not necessary to form the spherical auxiliary component described above.

The functional lens uses single-sided polished frosted glass, concave lens or planar lens.

The shape of the lens fixing frame is round, hexagonal or square, the area of the lens fixing frame is 2 mm²-100 mm², and the light emitting port fixture is round.

The number of the light emitting modules can usually be set as three, four or seven.

The invention further puts forward a device for use in medical treatment and cosmetology with laser, the device comprises power supply and control module, heat dissipation module, light source module and the contact-type laser operating head described above. The light source module uses a plurality of semiconductor lasers to emit light, which is respectively coupled to corresponding optical fiber, forming a plurality of paths of lasers, which are output to the corresponding light emitting modules.

A single-light-emitting unit semiconductor laser or the multi-light-emitting unit semiconductor laser, is a single-tube type semiconductor laser or a bar type semiconductor laser. The heat dissipation module can use an air cooling heat dissipation structure or a heat pipe heat dissipation structure.

The invention has the following advantages:

(1) A plurality of light emitting modules are used to form a light emitting port, on the whole, a large effective working area can be formed, the working efficiency is improved, meanwhile, every light emitting module can keep tight fit when being in contact with irregular surface, the risk of light leakage during the working process educed.

(2) When the invention is applied to laser medical cosmetology (such as laser hair removal), the single treatment area is increased, the treatment efficiency is enhanced, meanwhile, the skin can be tightly fitted, the risk of the light leakage during the treatment process is reduced, and the comfort degree is increased.

(3) A plurality of light source array is easily formed by using a single bar optical fiber module.

(4) Using optical fiber provides multiple choices to appearance design and beautification.

### BRIEF DESCRIPTION OF FIGURES

FIG. 1 schematically shows the structural diagram of the system according to this embodiment.

FIG. 2 schematically shows the diagram of a light emitting module contacting with skin.

FIG. 3 schematically shows the structural diagram of a light emitting module.

FIG. 4 schematically shows the structural diagram of another light emitting module.

FIG. 5 schematically shows a layout of three light emitting modules.

FIG. 6 schematically shows a layout of four light emitting modules.

FIG. 7 schematically shows a layout of seven light emitting modules.

FIG. 8 is a simplified schematic diagram of the whole optical system.

FIG. 9 schematically shows an air cooling heat dissipation structure.

FIG. 10 schematically shows a heat tube heat dissipation structure.

### DESCRIPTION OF LABELS IN THE ATTACHED FIGURES

1—skin; 2—functional lens; 3—optical universal head; 4—optical fiber; 5—lens fixing frame; 6—optical fiber fixture; 7—connector; 8—light emitting port fixture; 9—light source module; 10—focus lens.

### DETAILED DESCRIPTION

The invention is described in detail as follows, using the application in the aspect of laser medical cosmetology as an example.

As shown in FIG. 1, the whole system can be divided into light emitting module, optical system, heat dissipation module, power supply and control module and electrical power.

I. Light Emitting Module

1. Frosted glass: Light spot homogenization treatment is achieved by using single-sided polished frosted glass, the uniformity required by the specific technology needs to be satisfied. The surface contacted with the skin is required to be smooth and easy to slide.

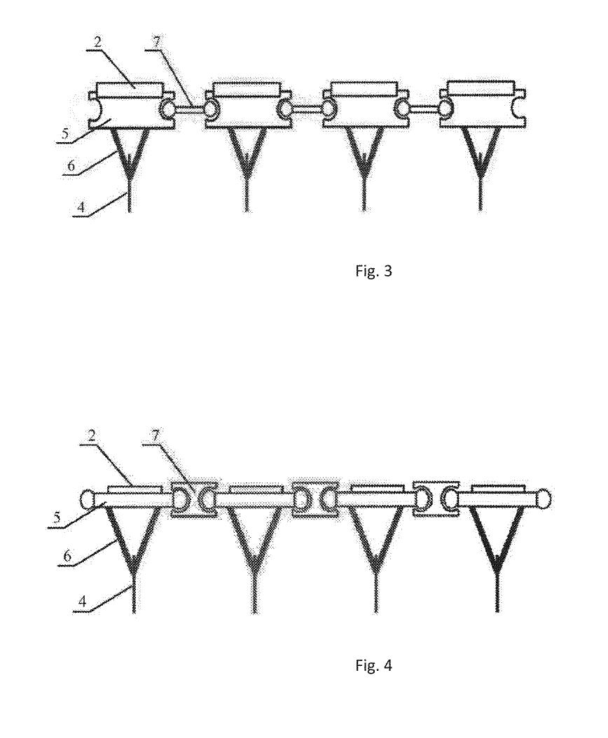

2. Optical universal head: As shown in FIG. 3, the frosted glass lens is fixed to the optical direction head, the optical fiber output end is fixed at a certain position behind the lens. Three or more optical direction heads are arranged on the same plane, and are mounted on the integral supporting device. Adjacent optical universal heads are connected using the connectors with spherical structure, every optical universal head can keep relative movement. The pressure due to the usage can cause the optical universal head to be rotated, driving the lens to rotate, so that the skin can be tightly fitted at the angular corner locations of the skin.

2.1 Lens: a single-sided polished frosted glass can be used to homogenize light spots, an upper concave mirror or a convex mirror, a plane lens and other lenses can also be mounted to achieve different functions.

2.2 Lens fixing frame: it should usually be made of the materials with relatively large strength, the frame body is a round hollow frame body, and a spherical concave hole is formed at the position where the connectors are required for fixing purpose at the periphery, to mount the spherical switching parts of the two ends of the connector. A sinking groove is formed at the position where the lens is mounted, so that the lens can be conveniently mounted. At the rear end of the lens fixing frame, there is a connecting port to mount the optical fiber fixture.

2.4 Optical fiber fixture: The frame to fix an optical fiber needs to connect a lens fixing frame and a fixed optical fiber light emitting port, maintain the light-light beam outputs of the optical fibers to be perpendicular to the mirror surface. A metal conical frame body, a high-strength spring and a high-strength elastic material can be used.

2.5 Connector: The connector can be a metal fixing body, the two ends are in a spherical shape, the spherical bodies at the two ends can also be connected through a connecting rod, which is made of high-strength elastic materials.

In the structure shown above in FIG. 3, the connector and the specific structure form of the lens fixing frame can be interchanged, as shown in FIG. 4.

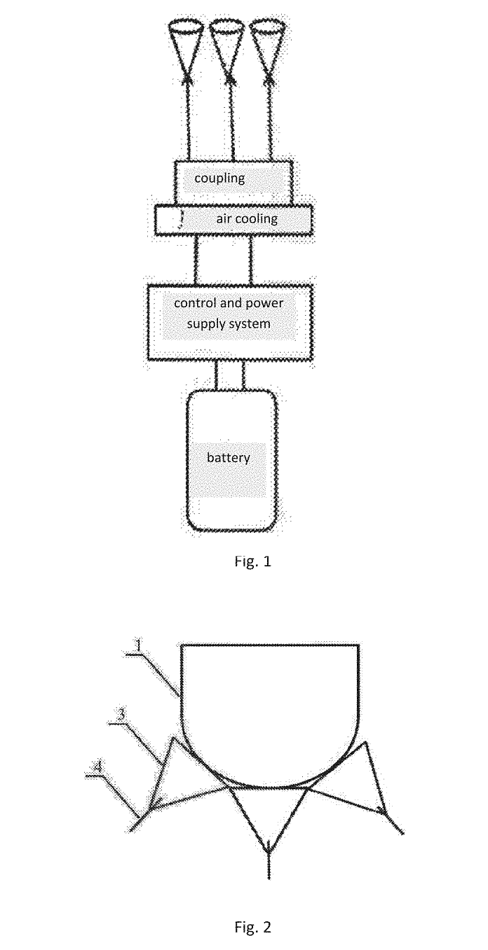

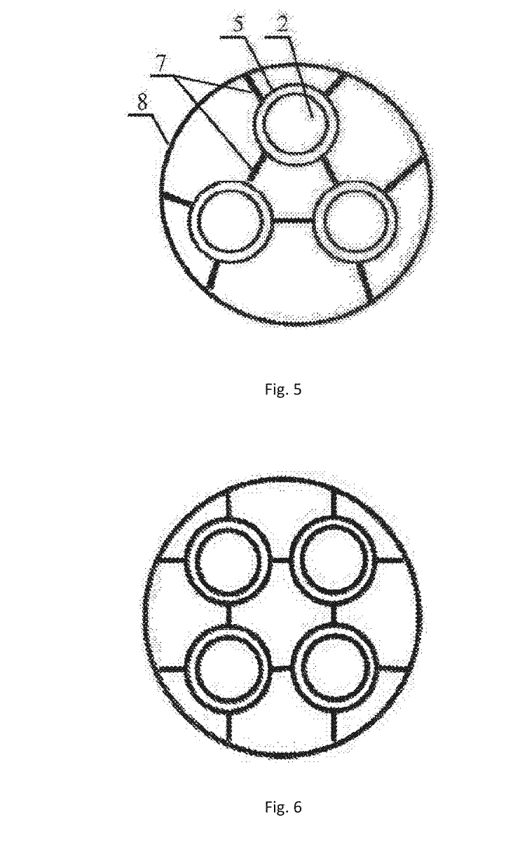

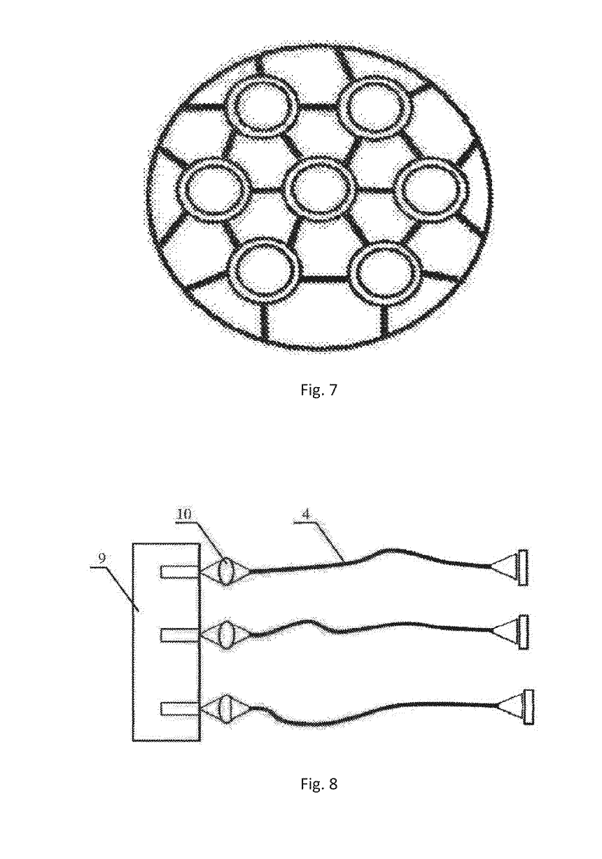

A plurality of light emitting modules are arranged, connector is connected with each other, and the edge is connected to the light emitting port fixture through connectors shown in FIG. 5, FIG. 6 and FIG. 7). So that every light emitting module is subjected to the pressure of the skin angular corner, and the corresponding deflection is made, the effect of fitting to the skin closely is achieved, as shown in FIG. 2.

In this way, the arrangement requirement of any number of light emitting ports can be satisfied.

II. Optical System

As shown in FIG. 8, plurality of single tube type laser light sources, coupled with a plurality of optical fibers, are used to form a plurality of paths of laser, which are output to the lens. A plurality of paths of lasers can also be formed by coupling a bar type laser light source into a plurality of optical fibers, the output power of every optical fiber ranges from 5 to 10 w.

Laser light source: It can be C-mount type, F-mount type single-tube plus fiber coupling, single-tube optical fiber coupling series products, can also be a bar-type plus optical fiber coupling product.

III. Heat Dissipation Module

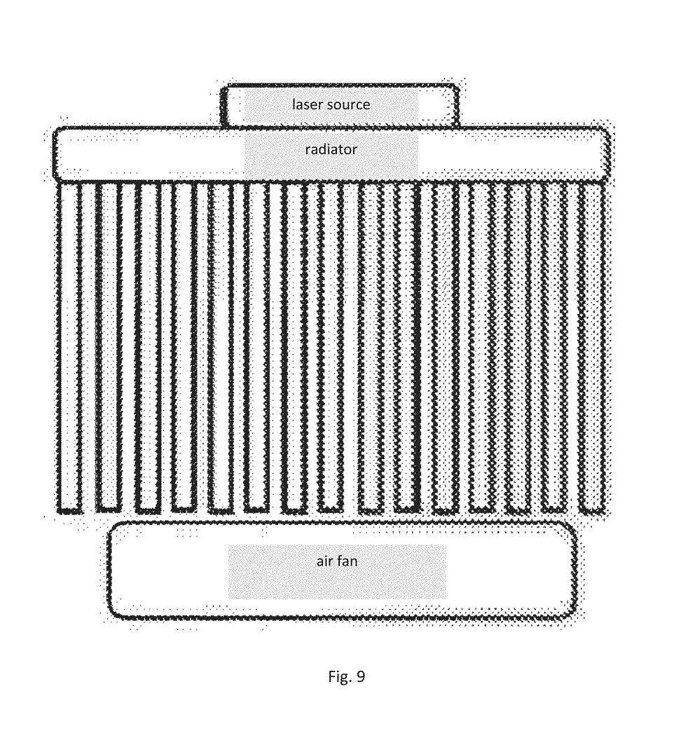

The laser light source is mounted on the radiator, the cooling is conducted with the radiator, as shown in FIG. 9. The heat dissipation amount of the light source is used to select a proper radiator (the material and size of heat dissipation, the air outlet volume of the fan, etc.)

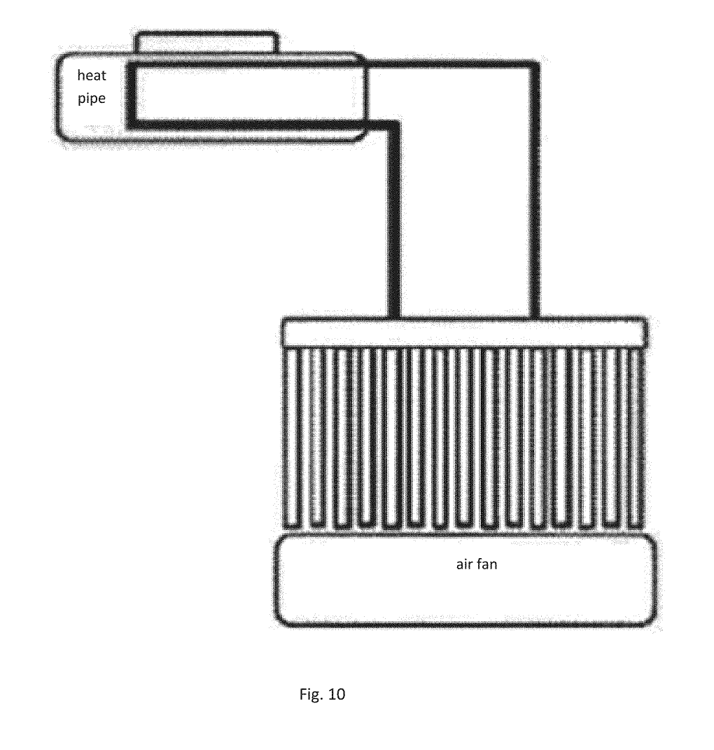

If the air cooling heat dissipation cannot meet the heat dissipation requirement of the light source, the heat pipe can also be used as a heat dissipation module, as shown in FIG. 10, its heat dissipation capacity is better than that of the heat dissipation fin.

What is claimed is:

1. A contact-type laser operating head, comprising a light emitting port fixture, and a plurality of light emitting modules forming a light emitting port; wherein a peripheral light emitting module on a periphery of the plurality of light emitting modules and the light emitting port fixture as well as the adjacent light emitting modules are configured to be connected by connectors; wherein the connectors or an adaptive connecting structure thereof enables each of the plurality of light emitting modules to be driven to rotate independently so as to follow and fit a to-be-processed surface; wherein the light emitting module comprises a lens fixing frame and an optical fiber fixture, wherein the lens fixing frame is configured to be used to mount a functional lens, and the optical fiber fixture is configured to be fixedly mounted behind the lens fixing frame, for packaging a coupling optical fiber and keeping a light beam output from the optical fiber to be perpendicular to the mirror surface.

2. The contact-type laser operating head of claim 1, wherein the optical fiber fixture is a conical frame structure, and an optical fiber coupling interface is arranged on a conical top.

3. The contact-type laser operating head of claim 1, wherein a corresponding connecting part between the connector and the light emitting port fixture, and a corresponding connecting part between the connector and the light emitting module, both form spherical auxiliary components.

4. The contact-type laser operating head of claim 3, wherein a main body of the connector is a rigid connecting rod; wherein each of two ends of the rigid connecting rod is a spherical part; wherein a spherical concave hole, arranged in a periphery of the lens fixing frame of the light emitting module, is connected with the spherical part in an adaptive mode; or, the main body of the connector is a rigid connecting block; wherein each of the two ends of the rigid connecting block is a spherical concave hole; wherein a spherical connecting head which extends out from an outer periphery of the lens fixing frame of the light emitting module is connected with the spherical concave hole in an adaptive mode.

5. The contact-type laser operating head of claim 1, wherein the connector is a connecting rod made of a high-strength elastic material, and the intensity and the elasticity meet independent driven rotation of each of the light emitting modules.

6. The contact-type laser operating head according to any one of claims 1 to 5, wherein the functional lens use single-sided polished glass, a concave lens or a planar lens.

7. The contact-type laser operating head according to any one of claims 1 to 5, wherein the shape of the lens fixing frame is round, hexagonal or square, and an area of the lens fixing frame is 2 mm²-100 mm²; wherein the light emitting port fixture is round.

8. The contact-type laser operating head according to any one of claims 1 to 5, wherein there are three, four or seven light emitting modules in total.

9. A device for use in medical treatment and cosmetology with laser, comprising a power supply and a control module, a heat dissipation module, a light source module and the contact-type laser operating head of the claim 1; wherein the light source module uses a plurality of semiconductor lasers to emit light, which is respectively coupled to a corresponding optical fiber, forming a plurality of laser outputs to corresponding light emitting modules.

10. The device for use in medical treatment and cosmetology with laser of claim 9, wherein a semiconductor laser of the plurality of semiconductor lasers is a single-light-emitting unit semiconductor laser or a multi-light-emitting unit semiconductor laser; wherein the heat dissipation module uses an air cooling heat dissipation structure or a heat pipe heat dissipation structure.
